(19) United States
(12) Patent Application Publication
(10) Pub. No.: US 2016/0128553 A1
(43) Pub. Date: May 12, 2016

(54) Intra- Abdominal Lightfield 3D Endoscope and Method of Making the Same

(71) Applicant: Zheng Jason Geng, Vienna, VA (US)

(72) Inventor: Zheng Jason Geng, Vienna, VA (US)

(21) Appl. No.: 14/535,336

(22) Filed: Nov. 7, 2014

(51) Int. Cl. A61B 1/05; A61B 1/06; A61B 1/00

(52) U.S. Cl. CPC A61B 1/05; A61B 1/00016; A61B 1/06; A61B 2576/00

(57) ABSTRACT

The inventions disclosed herein are related to various designs of intra-abdominal three-dimensional (3D) imaging systems that are able to provide 3D visualization, measurement, registration and display capability for minimally invasive surgeries.

The application is based on the provisional U.S. application No. 61/901,279, filed with United States Patent & Trademark Office on Nov. 7, 2013, entitled “Intra-Abdominal Lightfield 3D camera and Method of Making the Same”.

### 1. FIELD OF INVENTION

We disclose various designs of intra-abdominal three-dimensional (3D) imaging systems that are able to provide 3D visualization, measurement, registration and display capability for minimally invasive surgeries.

### 2. SUMMARY OF INVENTION

This invention discloses a novel lightfield 3D endoscope for intra-abdominal minimally invasive surgery (MIS) applications. It is particularly suited for laparoendoscopic single-site surgery (LESS), Natural orifice translumenal endoscopic surgery (NOTES), and Robotic LESS (R-LESS) procedures. The miniature lightfield 3D endoscope consists of multiple sensors for real-time multiview lightfield 3D image acquisition, an array of LEDs for providing adequate illumination of targets, soft cable for extracorporeal power and video signal connection. The lightfield 3D endoscope can be positioned within peritoneal cavity via various means. For example, it can be attached to abdominal wall using stitches. It can be positioned using a set of magnets attached or embedded to the device, allowing for controlling its position/orientation by a set of extracorporeal magnets placed on the external abdominal wall. The lightfield 3D endoscope is inserted into peritoneal cavity via a single access port, then is navigated to desirable location for best capturing the surgical site. It does not occupy the access port after its insertion, leaving the access port to other surgical instruments. The lightfield 3D endoscope provides unprecedented true 3D image capability for various clinical applications in advanced minimally invasive surgeries, such as LESS, NOTES and R-LESS.

It has the following desirable features:
(i) Eliminate the problems of “tunnel vision” and slewed viewing angle of existing laparo/endoscopic imaging devices by attaching a 3D endoscope on abdominal wall nearby the surgical site, thus offering a full field of view (FOV) of surgical scene with proper viewing angle and without obscuring;
(2) Spare the often over-crowded access port: Traditional laparo/endoscope occupies precious space in the access port all the time, preventing simultaneous uses of other instruments from the same port. The over-crowded port may cause collisions of instruments. The disclosed lightfield 3D endoscope uses a thin and soft cable to supply power and transmit video signal, without needing the full occupancy of an access port;
(3) Maintain correct and stable spatial orientation: Orientations of intraperitoneal images are sometimes sideward or upside down, making it challenging for surgeons to establish a stable horizon and perceive depth during delicate surgical tasks. This can significantly increase surgeons' mental workload and degrade the efficiency and accuracy of LNR procedures. The disclosed lightfield 3D endoscope can be placed near surgical site leading to correct spatial orientation. Given its 3D imaging and processing capability, real-time images with correct orientation and viewing angle can always be presented for surgeons to view;
(4) Offer 3D depth cues: The lightfield 3D endoscope provides real-time 3D depth map, together with high resolution texture information, therefore can offer surgeons with enhanced 3D visual feedback in manipulating, positioning, and operating;
(5) Measure dimensions of surgical targets: lightfield 3D endoscope can offer quantitative dimensional measurements of objects in the scene, thanks to its unique 3D imaging capability;
(6) Perform image guided intervention (IGI): Lightfield 3D images facilitate accurate 3D registration between pre-operative CT/MRI data with in-vivo 3D surface data, thus enabling the IGI procedures.
(7) Glasses-free 3D display: The lightfield 3D images allow surgeons to visualize 3D target without using any special eyewear.

### 3. BRIEF DESCRIPTION OF THE DRAWINGS

FIG. 1 illustrates a lightfield 3D endoscope for intra-abdominal imaging applications.

FIG. 2 illustrates lightfield representation of the image stack captured by the lightfield 3D endoscope.

FIG. 3 illustrates a lightfield 3D endoscope with structured light illumination.

FIG. 4 illustrates a structured light 3D imaging method.

FIG. 5 illustrates an example of structured light illumination projector

FIG. 6 illustrates an exemplary design of multispectral and polarizing lightfield 3D endoscope

FIG. 7 illustrates a stereo imaging sensor.

FIG. 8 illustrates a wireless lightfield 3D endoscope design.

FIG. 9 illustrates an extracorporeal magnetic controller for anchoring and maneuvering the internal lightfield 3D endoscope.

FIG. 10 illustrates an example of direction control by extracorporeal magnetic controller.

FIG. 11 illustrates 3D processing algorithms and software architecture for the lightfield 3D endoscope.

### 4. DETAIL DESCRIPTION OF THE INVENTION

### 4.1. Background

Minimally invasive surgeries (MIS) are procedures in which devices are inserted into human body through natural openings or small skin incisions to diagnose and treat/repair a wide range of medical conditions as an alternative to traditional open surgeries. MIS has achieved pre-eminence for many general surgery procedures over the past two decades and has led to reduced risk of complications, faster recovery with enhanced patient satisfaction due to reduced postoperative pain and favorable health system economics.

To push the technical boundaries and further reduce morbidity of MIS, laparoendoscopic single-site surgery (LESS) technique was developed to minimize the size and number of abdominal ports/trocars. LESS has been used in cholecystectomy, appendectomy, adrenalectomy, right hemicolectomy, adjustable gastric-band placement, partial nephrectomy and radical prostatectomy. Compared with conventional laparoscopy, LESS procedures utilize single access port, and has clear benefits in terms of cosmetics, less postoperative pain, faster recovery, less adhesion formation, and shortened convalescence.

Natural orifice translumenal endoscopic surgery (NOTES) represents another recent paradigm shift in MIS fields. NOTES are performed with an endoscope passed through a natural orifice (mouth, urethra, anus, etc.) then through an internal incision (in stomach, vagina, bladder or colon) to access the disease site, thus altogether eliminating abdominal incisions/external scars. NOTES were used in human for diagnostic peritoneoscopy, appendectomy, cholecystectomy, and sleeve gastrectomy.

Robotic systems such as the da Vinci robotic system have been used for LESS, dubbed R-LESS, to provide easier articulation, motion scaling, and tremor reduction.

Despite the rapid expansion of these three major MIS advances (LESS, NOTES, and R-LESS (LNR)) over the past a few years, lack of proper LNR-specific instruments represents one of major technical hurdles that prevent a widespread adaptation of these new techniques, thus falling short in translating LNR's tangible benefits to more patients. The operation of LNR requires a single port access to the peritoneal cavity. This feature leads to a raft of broad challenges, ranging from the risk of instruments collisions (i.e., the “sword fight”) and difficulties in obtaining adequate traction on tissues for dissection, to the reduced triangulation of instruments.

Particularly, visualization capability of existing devices for LNR proves problematic and inadequate, since surgeons are no longer looking directly at the patient anatomy, but rather at a video monitor that is 2D and not in the direct hand-eye axis and the access port may not have direct view of the surgical site. Main drawbacks of these existing imaging devices include:
(i) Tunnel vision: The field of view (FOV) of laparoscopic images in LNR can be obscured or blocked by surgical devices that pass through the same access port.
(2) Full-time Occupancy of access port: Traditional laparo/endoscope occupies the precious space in access port all the time, preventing simultaneous uses of other instruments from the same port
(3) Instrument collisions: Occupancy of access port of laparo/endoscope may cause collision with other tools.
(4) Skewed viewing angle: placing a camera through the solitary port site in LNR procedures can create unfamiliar viewing angles, especially in NOTES [24].
(5) Difficulty in maintaining correct and stable spatial orientation: Orientations of intracorporeal images are sometimes sideward or upside down, making it challenging for surgeons to establish a stable horizon and perceive depth during delicate surgical tasks. This can significantly increase surgeons' mental workload and degrade the efficiency and accuracy of LNR procedures.
(6) Lack of 3D imaging capability and depth cues: More importantly, the cameras presently used in LNR can only acquire 2D images that lack the third dimension (the depth) information.
   The disclosure of this invention, therefore, is a novel lightfield 3D endoscope for MIS. It is also particularly suited for performing LESS, NOTES, and R-LESS procedures.

### 4.2. Lightfield 3D endoscope

### Embodiment #1

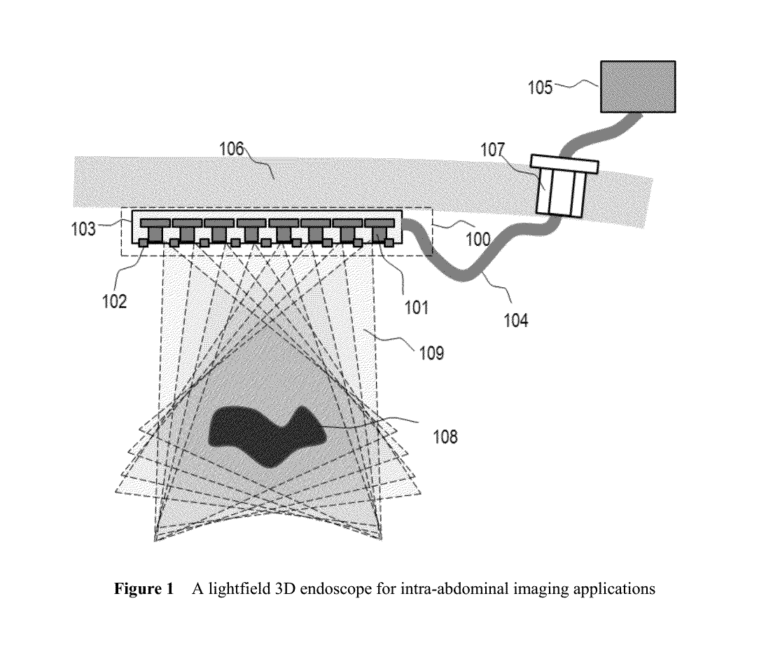

FIG. 1 illustrates an example design of the disclosed lightfield 3D endoscope 100. It consists of an array of imaging sensors 101, illumination devices 102, outer housing 103, connection cable 104 and extra-peritoneal control unit 105. Typical imaging sensors include charge-coupled device (CCD) or complementary metal-oxide-semiconductor (CMOS) sensors, but any other types of imaging sensor can be used. Both analog and digital version of CCD/CMOS sensor modules can be used. In an exemplary design, we select a CMOS chip from OmniVision, which has image resolution of 672×492 pixels, image area 4.032 mm×2.952 mm, and pixel size 6×6 μm. The high quality miniature optical lens are used which offers proper field of view (FOV) (for example 120-degree FOV). The geometric locations of all sensors are arbitrary but are known or can be obtained via calibration techniques. Sensors in the array can be all the same or differ in optical, mechanical and/or electronic characteristics. For example, these sensors can be of different focal lengths, field of view, spectrum range, pixel resolution or any other performance index. Both image and non-image signals can be acquired from these sensors.

The lightfield 3D endoscope 100 also includes one or more illumination device(s) 102. Typically, Laser Emitted Diodes (LED) are used, but any other means (such as light fiber) to provide proper illumination can also be used. In an exemplary design, we used mini-LEDs produced by Nichia Corp. The brightness of LEDs is user controllable. One or more cables 104 are used to provide power and single communications to and from the lightfield 3D endoscope 100 to extra-peritoneal control unit 105. The lightfield 3D endoscope 100 is inserted into intra-peritoneal cavity via an access port 107, and placed near the abdominal wall 106. The tether cable 104 provides necessary power and signal communication connection to and from the lightfield 3D endoscope unit. Therefore, the lightfield 3D endoscope unit 100 itself does not occupy the access port all the times. Sensors in the camera array 101 acquire images of one or more targets 108 within their field of views 109. The images and signals acquired are transferred to extra-peritoneal control unit 105 for process and visualization.

Conventional 2D laparoscopes and/or endoscopes provide 2D image only, without 3D depth cues. Stereo endoscopes such as these used in Da Vinci robots offer two images of a target scene with slightly different perspective. Drawbacks of conventional stereo endoscopes include:

(1) Stereo images can only be viewed using special eyewear, or on a specially designed viewing console completely isolates the surgeon from the OR surrounding environment;
(2) There are occlusions in the scene where precise 3D reconstruction and measurement are impossible;
(3) Viewer(s) cannot freely change the viewing angle of a target without having to move the sensor, which is difficult to do during LNR operations;
(4) Stereo does not facilitate large screen, head-up, eyeglasses-free (autostereoscopic) and interactive 3D display, due to lack of sufficient number of acquired views.

With multiple high resolution imaging sensors, the disclosed lightfield 3D endoscope overcomes these above-mentioned drawbacks of traditional stereo endoscopes.

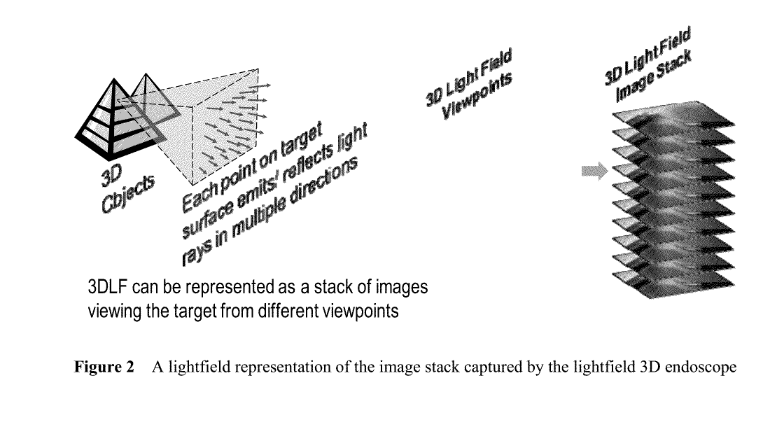

The complete 3D information (i.e., everything that can be seen) of the target 108 can be described by the lightfield. In computational lightfield acquisition literature, lightfield is often represented by a stack of 2D images, each viewing the target from different viewpoints. The captured images from the imaging sensor array 101 contain a rich set of light rays that are part of the lightfield generated by the target 108. In FIG. 2, lightfield is represented by a stack of multiple 2D images acquired by the lightfield 3D endoscope. The lightfield offers full resolution 2D/3D images, can facilitate 3D surface reconstruction, 3D measurement, and free-viewpoint visualization for glasses-free 3D display, among others. By processing the captured light rays, one can perform 3D surface reconstruction, rendering, and eye-glasses-free 3D visualization tasks.

Another key innovation of the lightfield 3D endoscope is to use a thin and soft tether cable 104 to provide power and video connection for the 100 module that can be easily navigated to a surgical site and positioned on abdominal wall. Advantages of this design are (1) By eliminating hard shaft of traditional laparoscopes/endoscopes, we can free-up the precious space in the access port for other surgical instruments and avoid the “sword fight”; (2) The lightfield 3D endoscope module 100 can be placed anywhere within the peritoneal cavity, not restricted by any shaft-related constraints. Commonly, we can place the 100 unit near a surgical site to have a “stadium view”, and to avoid the “tunnel vision” and skewed viewing angle, even the site is far away from the access port.

### 4.3. Embodiment #2

### Structured Light Lightfield 3D Endoscope

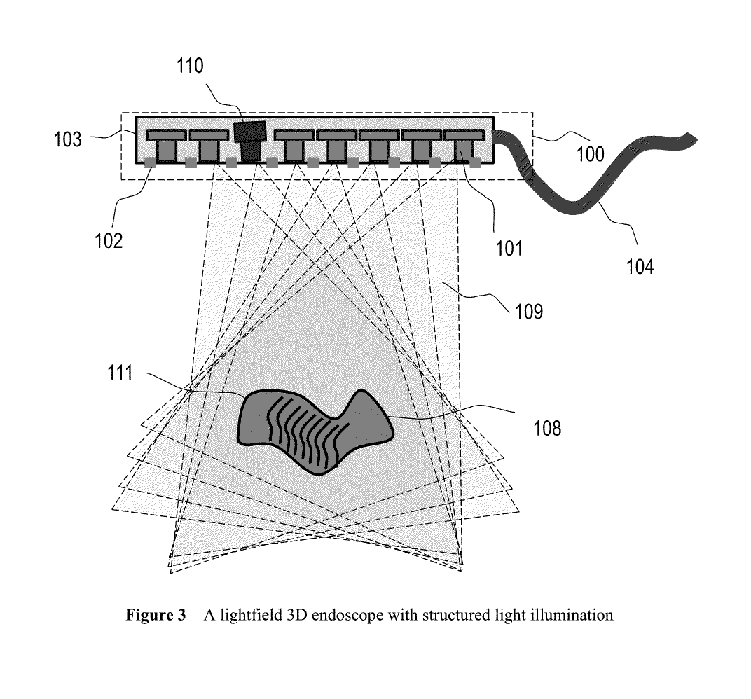

FIG. 3 discloses a design of the lightfield 3D endoscope with active structured light illumination mechanism. The structured light projector 110 generates spatially varying illumination pattern 111 on the surface of target 108. Structured light is a well-known 3D surface imaging technique. In this invention, we apply the structured light illumination technique to the lightfield 3D endoscope.

With projected surface pattern in by the structured light projector 110, one can easily distinguish surface features in the captured lightfield images. Reliable 3D surface reconstruction can be performed based on multiview 3D reconstruction techniques. This type of computation does not require calibrated geometric position/orientation of the structured light projector. The projected surface pattern only serve the purpose of enhancing surface features thus improve the quality and reliability of the 3D reconstruction results.

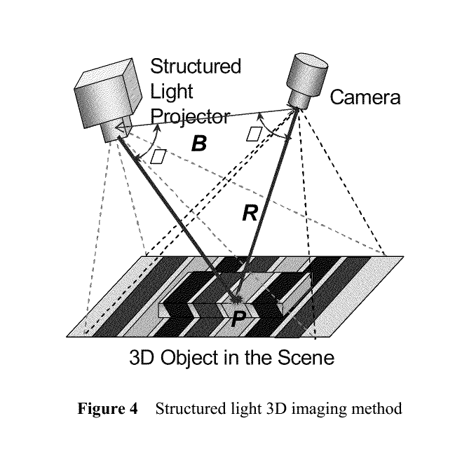

3D surface reconstruction can also be performed using structured light projection from a calibrated projector. In this case, the geometric information (position/orientation) of the structured light projector is known via precise calibration. FIG. 4 shows an example of such system with one imaging sensor, without loss of generality. The principle can be extended to systems with multiple imaging sensors and/or multiple structured light projectors. The geometric relationship between an imaging sensor, a structured light projector, and an object surface point can be expressed by the triangulation principle as:

$$R = B \frac{sin β}{sin ( α + β )}$$

The key for triangulation based 3D imaging is the technique used to differentiate a single projected light spot from the acquired image under a 2D projection pattern. Structured light illumination pattern provides a simple mechanism to perform the correspondence. Given known baseline B and two angles α and β, the 3D distance of a surface point can be calculated precisely.

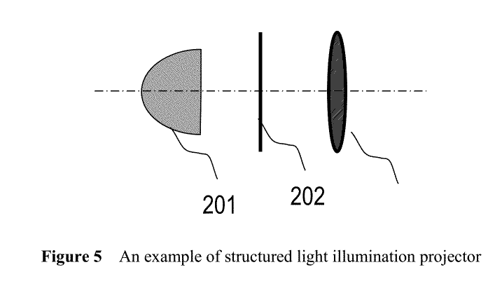

The miniature structured light projector 110 can be design in various forms. FIG. 5 illustrates an example of typical design. Light source 201 provides sufficient illumination of a pattern screen 202. An objective lens 203 project the image of the pattern screen towards the surface of target in the scene. Light source 201 can be an incoherence light source such as LED or fiber illuminator. The pattern on the pattern screen 202 is designed based on structured light (single shot) principle. The objective lens can be a multiple lens optical system that generates quality pattern projection.

The light source 201 can also be coherent such as laser. The pattern screen 202 can be a diffractive optical element (DOE), which is designed to have certain diffraction pattern. Such diffraction pattern can be used as the structured light illumination pattern. The miniature structured light projector can be designed using a miniature diffractive optical element (DOE), a GRIN collimator lens, or a single-mode optical fiber that deliver light from a light source. The projected pattern provides unique markers on target surface. 3D surface profile can then be obtained by applying triangulation algorithms.

### 4.4. Embodiment #3

### Multi-Spectral and/or Polarizing Lightfield 3D Endoscope

Given multiple imaging sensors on the lightfield 3D endoscope, one can configure some of sensors to acquire images in different spectral bands or different polarization directions. For example, narrow band filters can be used to enhance contrast (signal to noise ratio) of issue imaging. Polarizing imaging acquisition can suppress the effect of surface reflection on imaging quality.

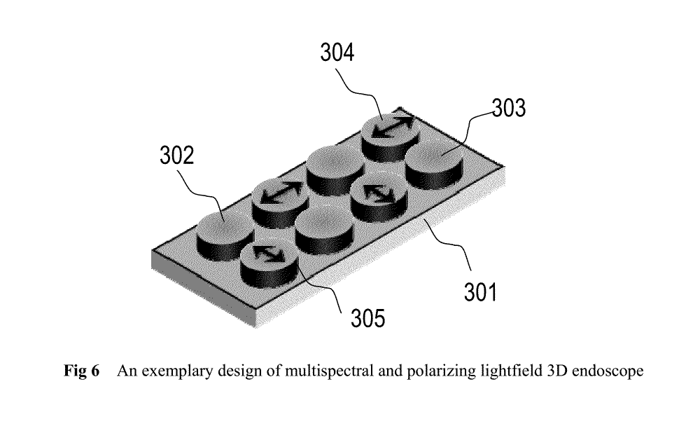

FIG. 6 illustrates an example of a mixed sensor platform with both spectral and polarization image capture channels. Note that the spectral imaging and polarization imaging are entirely independent imaging modalities. They can be used simultaneously, or separately, depending on specific application needs.

As shown in FIG. 6, there are eight optical channels; each has its unique spectral and polarization properties. They are used to acquire multi-spectrum composite images of target surface and sub-surface. The 3D surface profile can be reconstructed from any or all pairs of images acquired.

### 4.5. Embodiment #4

### Stereo Endoscope

In a design of lightfield 3D endoscope where only two imaging sensors are used, the system becomes stereo endoscope. This stereo endoscope design differs from conventional stereo endoscope in that its viewing angle is side-view.

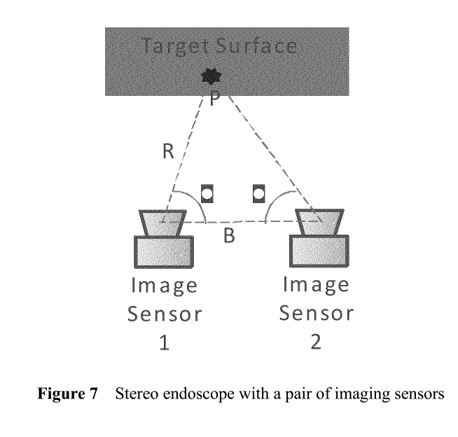

This 3D image acquisition technique is based on a pair of imaging sensors to acquire binocular stereo images of the target scene in a manner similar to human binocular vision, thus providing the ability to capture 3D information of the target surface (FIG. 7). The correspondence algorithms are developed to find accurate match of the same surface point P on both images. The geometric relationship between two image sensors and an object surface point P can be expressed by the triangulation principle as:

$$R = B \frac{sin β}{sin ( α + β )}$$

where B is the baseline between the two image sensors and R is the distance between the optical center of an image sensor and the surface point P. The (x,y,z) coordinate values of the target point P can then be calculated precisely based on the R, α, β, and geometric parameters.

### 4.6. Embodiment #5

### Wireless Lightfield 3D Endoscope

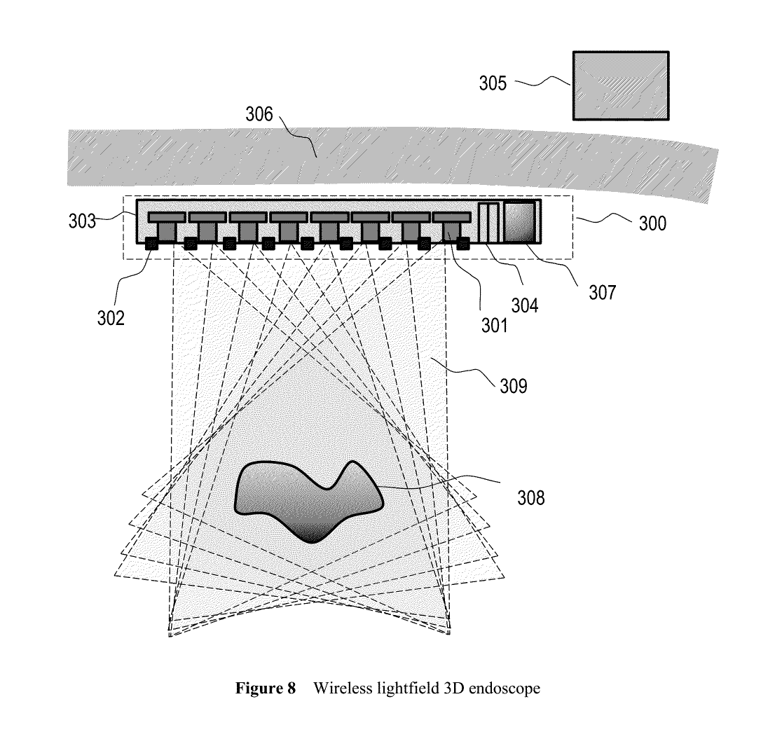

FIG. 8 illustrates a wireless lightfield 3D endoscope design. It has similar features as the one shown in FIG. 1, except that the tether cable 104 is eliminated. Instead, the wireless unit 300 carries a set of battery 304 for supplying the power of the self-contained wireless lightfield 3D endoscope 300, and a wireless communication link module 307 for transfer image signal acquired by the sensor array 301 to an extra-peritoneal wireless communication link unit 305. The battery 304 can be any types of miniature battery unit, such as lithium battery, as long as the battery capacity is sufficient to sustain the normal operation of the wireless lightfield 3D endoscope. The wireless communication link module is able to handle multi-channel image data transmission at a speed sufficient for clinical applications.

### 4.7. Embodiment #6

### Lightfield 3D Endoscope with Magnetic Guidance

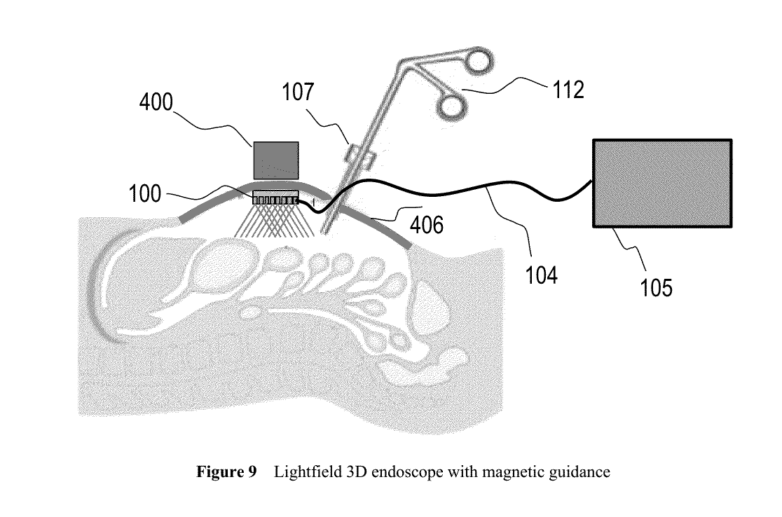

Another embodiment of the lightfield 3D endoscope is its magnetic anchoring and maneuvering mechanism, as illustrated in FIG. 9. The lightfield 3D endoscope unit 100 is augmented with embedded magnets or magnetic components. An extracorporeal magnetic controller (MC) 400 is used to attract the internal unit 100, and force it to attach on abdominal wall 406. The external MC can be moved by surgeon to desired locations, thus dragging the internal unit 100 to that desirable location. To ensure sufficient magnetic attraction forces, high grade magnet (such as nickel plated Neodymium (NdFeB) magnets (grade 52)) may be used. They need to ne magnetized in proper direction.

Comparing with various self-propel robotic driving mechanisms, use of passive magnets for anchoring and maneuvering internal imaging sensor has several advantages: (1) Simple and low-cost; (2) compact; (3) light weight; (4) no active components thus no power supply is needed; (5) reliable and fail-safe.

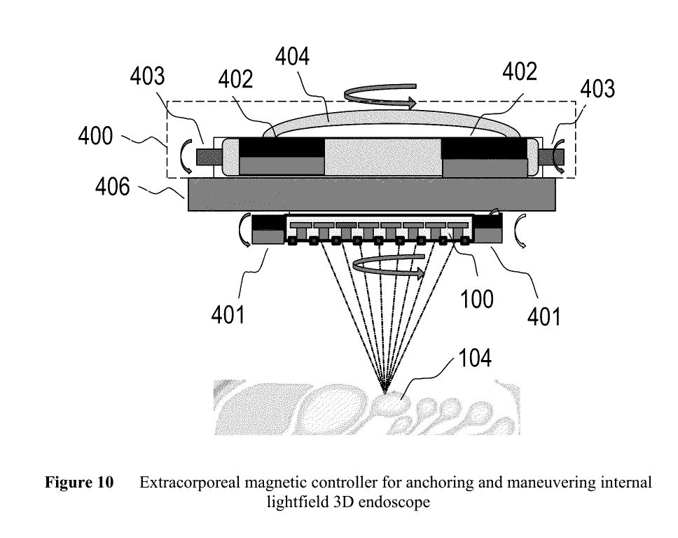

The details an exemplary design of the MC unit is illustrated in FIG. 10. The lightfield 3D endoscope unit 100 is augmented with a pair of magnets 401. In the extra-peritoneal MC unit 400, there are pairs of magnets 402 configured to generate magnetic force to attract the intra-peritoneal magnets 401. To control the position and orientation of the intra-peritoneal lightfield 3D endoscope 100 with magnets 401, one can move the extra-peritoneal MC unit 400, which generates sufficient magnetic force to drag the intra-peritoneal unit 100 and 401 to the desirable position and orientation.

The design shown in FIG. 10 is also able to control the axial rotation of the intra-peritoneal unit. An axial rotation mechanism 403 is built into the mounting of magnets 402. Operator can manually (or electronically) control the axial rotation of magnet 402. The rotation of 402 leads to changes in direction of the magnetic field, which exert rotation forces to the pair of intra-peritoneal magnets 401, thus generating rotation motion for lightfield 3D endoscope 100.

A handle 404 is shown in FIG. 10 to illustrate proper way to operate the MC unit 400. Any other type of designs may achieve the same purpose to provide secure and convenient way to move and rotate the MC unit.

### 4.8. Embodiment #7

### Software Processing Methods

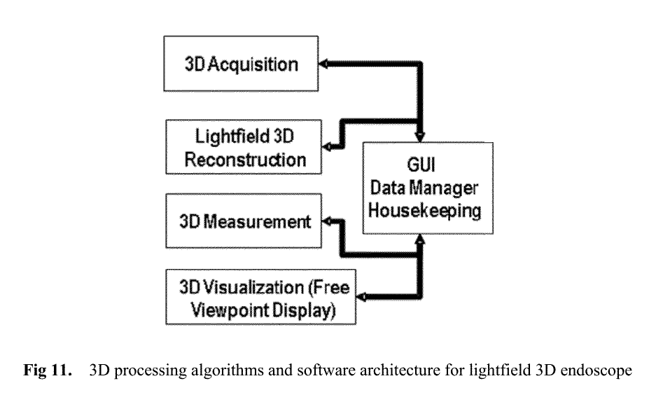

The operation of lightfield 3D endoscope system relies heavily on 3D image processing algorithms and software. FIG. 11 shows major software modules and processing method flowchart.

### 3D Acquisition:

This module controls the image acquisition operation. Since the lightfield 3D endoscope acquires multiple channel images simultaneously, acquisition control software should facilitate such simultaneous acquisition of high resolution full-color images without delay.

### Lightfield 3D Reconstruction:

Given multiple images acquired by the lightfield 3D endoscope, this software module carries out 3D surface reconstruction to obtain digital 3D profile of target surface.

### 3D Measurements:

With reconstructed 3D surface data, this software module perform quantitative 3D measurements, such as distance between selected points, area, and volume of selected target.

### Free Viewpoint 3D Visualization:

With acquired lightfield information, this software module enables real-time display of lightfield 3D data and facilitates true free viewpoint 3D visualization of target from any desirable viewing perspective, viewing angle, and without requiring any special eyewear. Viewers can change his/her eyes position to see different perspectives from different viewing angles. There is no restricted viewing zone to confine the operator. This provides significant advantage to practical clinical MIS operators.

### GUI, Data Management and Housekeeping Functions:

This module perform all necessary GUI/data-management/housekeeping functions to enable effective and efficient operations and visualization of the lightfield 3D endoscope.

The methods and systems of certain examples may be implemented in hardware, software, firmware, or combinations thereof. In one example, the method can be executed by software or firmware that is stored in a memory and that is executed by a suitable instruction execution system. If implemented in hardware, as in an alternative example, the method can be implemented with any suitable technology that is well known in the art.

The various engines, tools, or modules discussed herein may be, for example, software, firmware, commands, data files, programs, code, instructions, or the like, and may also include suitable mechanisms.

Reference throughout this specification to “one example”, “an example”, or “a specific example” means that a particular feature, structure, or characteristic described in connection with the example is included in at least one example of the present technology. Thus, the appearances of the phrases “in one example”, “in an example”, or “in a specific example” in various places throughout this specification are not necessarily all referring to the same example. Furthermore, the particular features, structures, or characteristics may be combined in any suitable manner in one or more examples.

Other variations and modifications of the above-described examples and methods are possible in light of the foregoing disclosure. Further, at least some of the components of an example of the technology may be implemented by using a programmed general purpose digital computer, by using application specific integrated circuits, programmable logic devices, or field programmable gate arrays, or by using a network of interconnected components and circuits.

Connections may be wired, wireless, and the like.

It will also be appreciated that one or more of the elements depicted in the drawings/figures can also be implemented in a more separated or integrated manner, or even removed or rendered as inoperable in certain cases, as is useful in accordance with a particular application.

Also within the scope of an example is the implementation of a program or code that can be stored in a machine-readable medium to permit a computer to perform any of the methods described above.

Modules may also be implemented in software for execution by various types of processors. An identified module of executable code may, for instance, comprise one or more blocks of computer instructions, which may be organized as an object, procedure, or function.

Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

While the forgoing examples are illustrative of the principles of the present technology in one or more particular applications, it will be apparent to those of ordinary skill in the art that numerous modifications in form, usage and details of implementation can be made without the exercise of inventive faculty, and without departing from the principles and concepts of the technology. Accordingly, it is not intended that the technology be limited, except as by the claims set forth below.

1. A 3D endoscope comprising:
an imaging unit and a control unit,
wherein the imaging unit comprises an outer housing and an array of imaging sensors and an illumination device located in the outer housing;
the array of imaging sensors include multiple imaging sensors for providing 2D images of the target captured under the illumination of the illumination device;
the control unit is configured for synthesizing a 3D image of the target with the 2D images of the target captured by each of the imaging sensors.

2. A 3D endoscope according to claim 1, further comprising:
soft cable, which is connected between the control unit and the imaging unit, for providing power supply to the imaging unit, and transmitting the multiple 2D images captured by the array of imaging sensors to the control unit.

3. A 3D endoscope according to claim 1, wherein:
the illumination device comprises a structured light projector;
the structured light projector is configured for generating structured pattern on the surface of the target;
each imaging sensor in the array of the imaging sensors is configured for capturing a 2D image of the structured pattern and transmitting it to the control unit;
the control unit is configured for making a 3D reconstruction of the target based on multiple 2D images of the structured pattern.

4. A 3D endoscope according to claim 3, wherein:
the structured light projector comprises a light source, a pattern screen and objective lens, the light source and the objective lens being located in two sides of the pattern screen;
the light source is configured for providing illumination for the pattern screen;
a preset image is on the pattern screen;
the objective lens is configured for projecting the light emitted from the light source and passing through the pattern screen on the surface of the target to generate the structured pattern on the surface of the target.

5. A 3D endoscope according to claim 1, wherein said array of imaging sensors comprises multiple image sensors with different spectral features and polarization features.

6. A 3D endoscope according to claim 1, wherein:
said array of imaging sensors comprises two imaging sensors, which are located on the two ends of the outer housing, for capturing 2D images of the target from a left-side perspective and a right-side perspective.

7. A 3D endoscope according to claim 1, further comprising a first wireless communication link module and a second wireless communication link module;
the first wireless communication link module is located in the imaging unit, and the second wireless communication link module is located in the control unit;
the first wireless communication link module is configured for transmitting the multiple 2D images captured by the array of imaging sensors to the second wireless communication link;
the imaging unit further comprises a set of battery for power supply to the imaging unit.

8. A 3D endoscope according to claim 1, further comprising a magnetic guidance means and a magnetic controller;
the magnetic guidance means is installed in the imaging unit and configured for driving the imaging unit to translate and/or rotate under control of the magnetic controller.

9. A 3D endoscope according to claim 1, further comprising:
a display unit, which is connected to the control unit, for displaying the 3D image of the target generated by the control unit.

10. A 3D imaging method comprising:
multiple imaging sensors in an array of imaging sensors capturing 2D images of a target under illumination provided by a illumination device;
a control unit synthesizing a 3D image of the target based on the 2D images of the target captured by each of the imaging sensors.

11. A 3D imaging method according to claim 10 further comprising:
a soft cable connected between the control unit and the imaging unit providing power supply to the imaging unit, and transmitting the multiple 2D images captured by the array of imaging sensors to the control unit.

12. A 3D imaging method according to claim 10, wherein the step of multiple imaging sensors in an array of imaging sensors capturing 2D images of the target under illumination provided by the illumination device further comprises:
a structured light projector in the illumination device generating structured pattern on the surface of the target;
each of the imaging sensors in the array of the imaging sensors capturing a 2D image of the structured pattern and transmitting it to the control unit;
and wherein the step of the control unit synthesizing a 3D image of the target based on the 2D images of the target captured by each of the imaging sensors further comprises:
the control unit making a 3D reconstruction of the target based on the multiple 2D images of the structured pattern.

13. A 3D imaging method according to claim 12, wherein the step of the structured light projector in the illumination device generating structured pattern on the surface of the target further comprises:
a light source providing illumination for a pattern screen;
a preset image is on the pattern screen;
objective lens projecting the light emitted from the light source and passing through the pattern screen on the surface of the target to generate the structured pattern on the surface of the target.

14. A 3D imaging method according to claim 10, wherein the step of multiple imaging sensors in an array of imaging sensors capturing 2D images of the target under illumination provided by the illumination device further comprises:
the multiple imaging sensors in the array of imaging sensors capturing the 2D images of the target with different spectral features and polarization features.

15. A 3D imaging method according to claim 10, wherein the step of multiple imaging sensors in an array of imaging sensors capturing 2D images of the target under illumination provided by the illumination device further comprises:
the array of imaging sensors include two imaging sensors;
the two imaging sensors in the array of imaging sensors capture 2D images of the target from a left-side perspective and a right-side perspective respectively.

16. A 3D imaging method according to claim 10, further comprising:
a first wireless communication link module located in the imaging unit transmitting the multiple 2D images captured by the array of imaging sensors to a second wireless communication link module located in the control unit.

17. A 3D imaging method according to claim 10, further comprising:
a magnetic guidance means installed on the imaging unity driving the imaging unity to translate and/or rotate under control of a magnetic controller.

18. A 3D imaging method according to claim 10, further comprising:
a display unit connected with the control unit displaying the 3D image of the target generated by the control unit.
